(19) United States
(12) Patent Application Publication
(10) Pub. No.: US 2009/0099644 A1
(43) Pub. Date: Apr. 16, 2009

(54) COVERED STENT

(75) Inventors: Youssef BIADILLAH, Sunnyvale, CA (US); Beshoy Guirguis, Mississauga (CA)

Correspondence Address: Ralph A. Dowell of DOWELL & DOWELL P.C., 2111 Eisenhower Ave, Suite 406, Alexandria, VA, 22314, US

(73) Assignee: Baylis Medical Company Inc., Montreal (CA)

(21) Appl. No.: 12/205,704

(22) Filed: Sep. 5, 2008

Related U.S. Application Data
(60) Provisional application No. 60/970,154, filed on Sep. 5, 2007.

(51) Int. Cl. A61F 2/82

(52) U.S. Cl. 623/1.16

(57) ABSTRACT

A stent to be used in an intraluminal cavity is disclosed. The stent comprises an elongated scaffold formed by a plurality of cells formed by a plurality of interlinked struts. In some embodiments, the scaffold comprises two longitudinally opposed ends, each of the ends having at least one protrusion that can be used to secure a cover to the stent. Each protrusion defines a cavity extending therethrough. The cover is secured to the scaffold at the protrusion by an adhesive extending through the cavity. Alternatively, the scaffold comprises at least two protrusions or segments, each defining a cavity for securing one end of the cover to the scaffold.

### CROSS-REFERENCES TO RELATED APPLICATIONS

This application claims the benefit of U.S. Provisional Patent Application Ser. No. 60/970,154 filed on Sep. 5, 2007, which is incorporated herein by reference in its entirety.

### TECHNICAL FIELD

The invention relates to an intravascular support device. More particularly, the invention relates to a stent that is to be used in an intraluminal space.

### BACKGROUND OF THE ART

A covered stent is disclosed in, for example, U.S. Pat. No. 6,626,939 issued to Burnside et al. on Sep. 30, 2003. This patent describes a stent graft comprising a cover formed of expanded Polytetrafluoroethylene (ePTFE) attached to a stent. The ePTFE is attached to the stent with the use of an adhesive application that is non-selective, such that the entire stent is sprayed with adhesive prior to the stent being thermally attached to the graft. Expansion of such a stent graft causes excessive strain in regions of the ePTFE cover that are not bonded to the stent, due to immobility of the ePTFE cover in the large areas that are bonded to the stent. This can cause tears to develop in the ePTFE cover in areas experiencing the greatest strain.

Another method of attaching an ePTFE cover to a stent is disclosed in U.S. Pat. No. 6,808,533, issued to Goodwin et al. on Oct. 26, 2004. The patent describes placement of an inner cover within the lumen of the stent structure and placing an outer cover over the stent structure. Either cover can be coated with an adhesive. A compression member is placed over the outer cover and the compressed covered stent is heated to bond the inner cover to the outer cover. When this stent-graft is expanded, tears may appear in the stent covering similar to those described above.

In U.S. Pat. No. 6,254,632, issued on Jul. 3, 2001 Wu et al. disclose an implantable medical device having protruding surface structures for drug delivery and cover attachment. Wu et al. teach protruding structures formed on a surface of the device that have a central depression region surrounded by a lip. The protruding structures can be used to help secure the cover, Glue can be added to the protruding structures to help secure the cover. The protruding structures can also contain a therapeutic substance or substances for release in-situ. Wu et al. disclose that the protruding structures will indent and deform but generally not puncture the cover. The indentations on the cover thus provide a rough outer surface which may increase the risk of restenosis after stent implantation.

Thus, it would be desirable to provide a novel stent structure for securing a cover thereto, as well as a method for securing the cover, that resolves one or more of the aforementioned deficiencies. These deficiencies have heretofore not been recognised in the art and have not been addressed by existing devices.

### BRIEF DESCRIPTION OF THE DRAWINGS

In order that the invention may be readily understood, embodiments of the invention are illustrated by way of examples in the accompanying drawings, in which:

FIG. 1 is a perspective view of the stent structure, in accordance with one embodiment of the present invention, depicting annular protrusions at the peripheral ends;

FIG. 2a is a top side view of a single cell unit forming the stent structure in the embodiment shown in FIG. 1;

FIG. 2b is a top side view of an embodiment of an annular protrusion that is structurally attached to a cell;

FIG. 3 is a sectional view of an embodiment of an annular protrusion, taken along the line 3-3 in FIG. 2b;

FIG. 4 is a top side view of a segment of a stent scaffold formed by a plurality of cells in accordance with one embodiment of the present invention;

FIG. 5 is a top side view of an alternate embodiment of a stent with annular protrusions attached to every other cell;

FIGS. 6a-6d are top side views of alternate embodiments of a scaffold segment joining two adjacent cells;

FIG. 7 is a perspective view of the stent in accordance with on embodiment of the present invention;

FIGS. 8a-c are top side views of alternate embodiments of protrusions;

FIG. 9a-c are top side views of further embodiments of protrusions;

FIG. 10a-c are top side views of still further embodiments of protrusions;

FIG. 11 is a perspective view of a covered stent in accordance with an embodiment of the present invention;

FIG. 12 is a perspective view of a cover secured to a stent using an embodiment of a method of the present invention; and

FIG. 13 is a cross-sectional view of a portion of a cover secured to a portion of a scaffold according to one embodiment of the present invention.

### DETAILED DESCRIPTION

Stents are often used to treat both vascular and non-vascular conditions within the body. In vascular conditions, stents are often used to treat stenosis, where the blood flow is restricted due to narrowing of the vessel at a length along the vessel. To improve blood flow in these areas, a stent can be placed to widen the vessel at the point of constriction. The stent can be surgically implanted within the patient in the expanded form or alternatively, the stent can be compressed and then inserted percutaneously and can then be deployed through balloon angioplasty. Currently available stents can cause tears along the vascular wall when in the expanded state. This can cause internal bleeding requiring surgery. Another complication that can arise due to the placement of the stent, is the formation of an aneurysm over time at the site of the stent where the vessel wall is in a weakened state. The vessel wall at this region can bulge outwards, and this region is subjected to increased amounts of pressure over time due to the flow of blood entering and leaving the vessel. Over time these areas can rupture causing internal bleeding. A way to avoid clinical complications arising from the placement of a standard stent, as well as further potential problems arising from tearing of the cover of presently available covered stents, is to provide a stent having a novel structure for enabling the stent to be covered with, for example, ePTFE.

Thus, in one broad aspect, the present invention comprises a stent comprising: a substantially elongated scaffold formed by a plurality of cells comprising a plurality of interlinked struts, said scaffold having two longitudinally opposed ends; at least two protrusions, at least one of said at least two protrusions being structurally attached to at least one of said plurality of cells at one of said two longitudinally opposed ends, and at least another of said at least two protrusions being structurally attached to at least one of said plurality of cells at another of said two longitudinally opposed ends; each of said at least two protrusions defining a cavity extending therethrough; and a cover attached to said scaffold by an adhesive, said adhesive being located within the cavities to secure said cover to said scaffold at the protrusions.

As a feature of this aspect the cover defines at least one hole, said at least one hole being aligned with at least one of said cavities, said adhesive extending through both said at least one of said cavities as well as said at least one hole.

In another broad aspect, the present invention comprises a stent comprising: a substantially elongated scaffold formed by a plurality of cells comprising a plurality of interlinked struts, said scaffold having two longitudinally opposed ends; a cover attached to said scaffold, and at least two protrusions or segments each structurally attached to at least one of said plurality of cells, at least one of said protrusions or segments for securing one end of said cover, at least another of said protrusions or segments for securing another end of said cover, each of said at least two protrusions or segments defining a cavity extending therethrough; wherein said cover is attached to said scaffold using an adhesive, said adhesive being located within the cavities to secure said cover to said scaffold at the protrusions or segments.

In one embodiment of the present invention, a covered stent is provided that has a smooth outer surface. This allows to minimize the risk of restenosis once the covered stent has been implanted into the patient's body.

Embodiments of the present invention allow for selective application of an adhesive to the stent and/or a cover. In some particular embodiments, the stent is structured to minimize the number of sites at which the cover may be secured, while maximizing adhesion at each of those sites. This allows for expansion of a covered stent where strain in areas of the cover between cells is minimized due to reduction in attachment points. This can minimize the risk of tears developing in the expanded cover.

With specific reference now to the drawings in detail, it is stressed that the particulars shown are by way of example and for purposes of illustrative discussion of certain embodiments of the present invention only. In this regard, no attempt is made to show structural details of the invention in more detail than is necessary for a fundamental understanding of the invention, the description taken with the drawings making apparent to those skilled in the art how the several forms of the invention may be embodied in practice.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

FIG. 1 illustrates one embodiment of a stent 1 of the present invention comprising a lattice or scaffold 8 made of a regularly repeating structural pattern or cell 4, composed of interlinked struts 5. The scaffold 8 has two longitudinally opposed ends 2 and 3. The cells 4 at each of the two longitudinally opposed ends are structurally attached to at least one protrusion 6. The protrusion 6 defines a cavity 7, extending substantially completely therethrough. In the embodiment shown in FIG. 1, the protrusions extend longitudinally from the two longitudinally opposed ends of the scaffold.

FIG. 2a shows a cell 4 of the embodiment of the stent shown in FIG. 1, made of interlinked struts 5. FIG. 2b illustrates a similar embodiment of a cell with the addition of a protrusion 6 structurally attached to the cell. In some embodiments the protrusion extends integrally from the cell. The protrusion defines at least one cavity 7 extending therethrough. In some embodiments, the protrusions do not extend integrally from the cells 4 but may be otherwise attached, for example by soldering, welding or other mechanisms of attachment. In some embodiments the protrusion has substantially rounded edges.

FIG. 3 shows a cross-sectional view through a protrusion 6 having a cavity 7 extending substantially through the entire protrusion. As will be described further hereinbelow, the cavity allows for a greater surface area for applying a bonding material such as an adhesive to the scaffold.

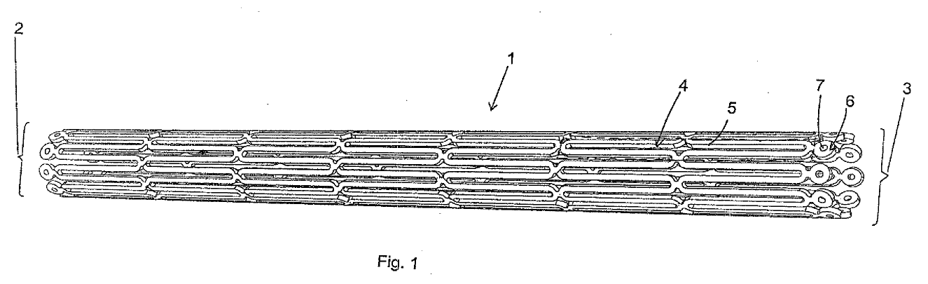

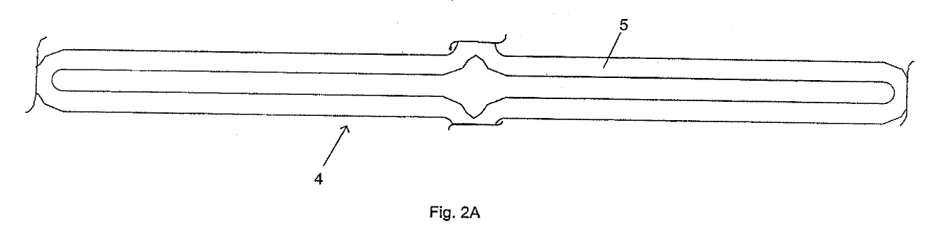

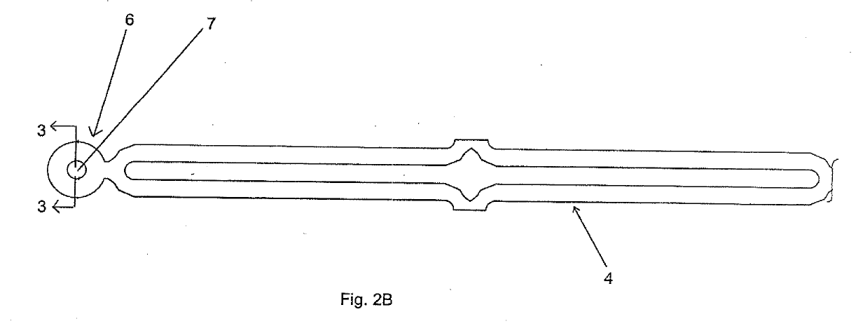

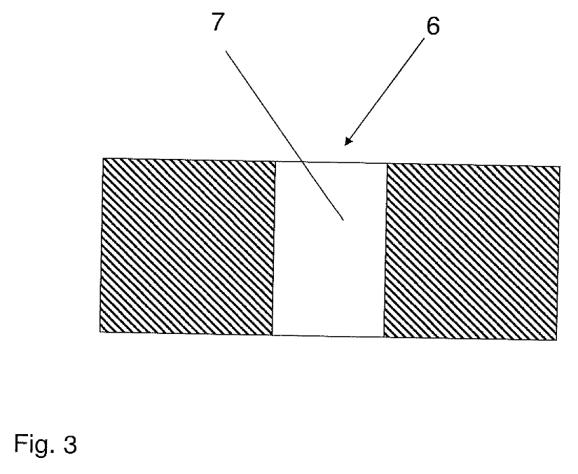

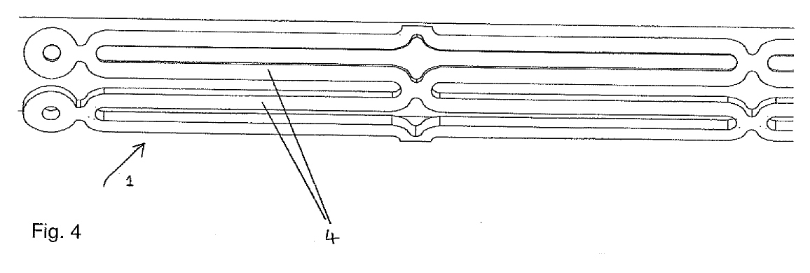

FIG. 4 illustrates a portion of the elongated scaffold structure of the stent 1 formed by a plurality of structurally interconnected cells 4.

In some embodiments of the present invention, as illustrated for example in FIG. 5, the cells 4 to which one or more of the protrusions 6 are structurally attached are circumferentially spaced apart from one another. In some such embodiments, the protrusions 6 are structurally attached to every second cell 4 at each of the longitudinally opposed ends. In alternative embodiments, the protrusions are spaced apart by a greater or lesser amount of cells.

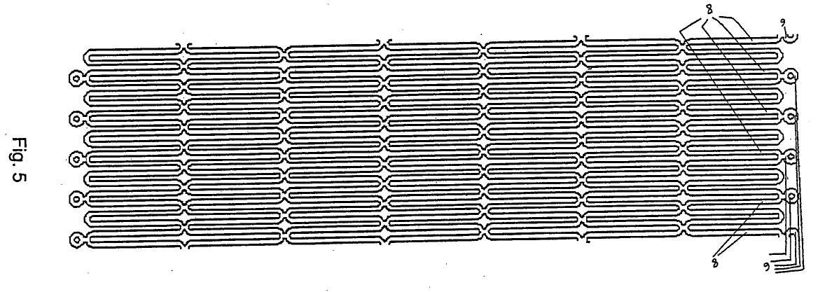

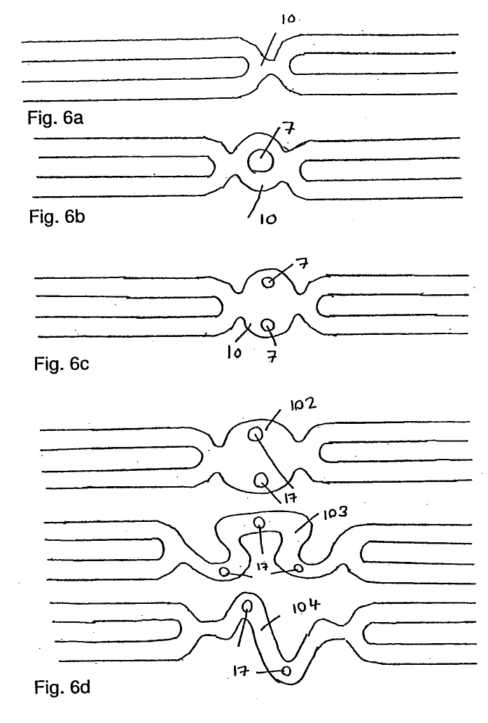

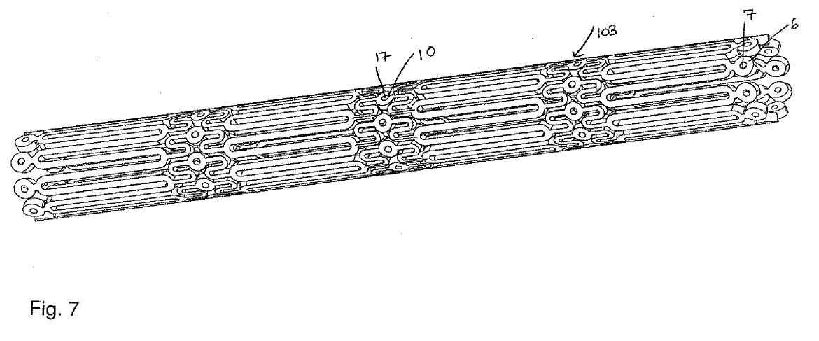

In some embodiments of the present invention, as illustrated for example in FIGS. 6a-6d, the stent 1 contains segments 10, disposed between two or more adjacent cells, for example between two or more longitudinally adjacent cells 4, within the scaffold. In the embodiment of FIG. 6a, the segment 10 is substantially straight. As illustrated in FIG. 6b, in another embodiment of the present invention, the segment 10, disposed between and forming the connection between two longitudinally adjacent cells, may define at least one cavity 17 extending therethrough. In one particular embodiment, segment 10 defines a plurality of cavities, as shown in FIG. 6c. In such embodiments, the cavity or cavities 17 defined by the segment 10 extend substantially completely therethrough, forming a channel through segment 10. In alternate embodiments of the present invention, as shown for example in FIG. 6d, the segment 10 may have a substantially disk-shaped configuration 102, a substantially ohm-shaped configuration 103 or a substantially sinusoidal configuration 104. Some such configurations allow for increased flexibility within segment(s) 10, which increases stent flexibility and can ease its navigation through tortuous anatomy. In any such embodiments, segment(s) 10 may each define at least one cavity 17, extending therethrough. FIG. 7 is a perspective view of an alternate embodiment of a stent 100, incorporating a plurality of segments 10 between longitudinally adjacent cells.

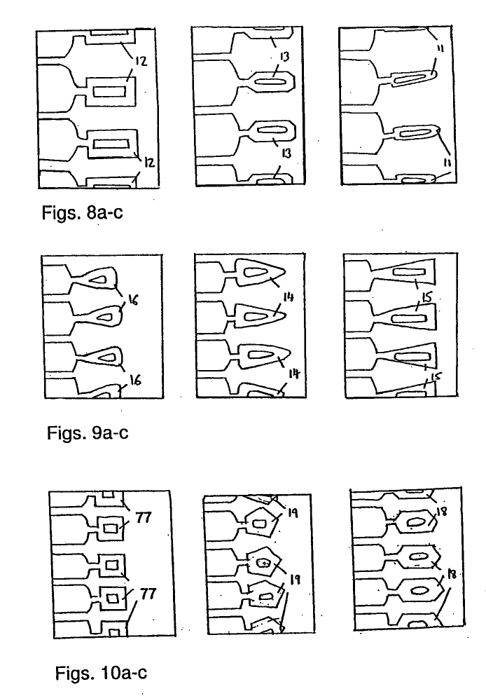

In some embodiments of the present invention, the protrusions 6 or segments 10 may be of various other structural shapes other than the substantially annular or disc shape illustrated in FIGS. 1-4. FIGS. 8a-8c, 9a-9c and 10a-10c, for example, illustrate different shape configurations of the protrusion. Similar variations can also be utilized for the shape of the segment and/or for the shape of the cavity defined by the segment. FIGS. 8a-8c generally illustrate elongated configurations of the protrusion. FIGS. 8 a-c illustrate oblong 11, rectangular 12 and octagonal configurations 13 of the protrusion respectively. FIGS. 9a-9c generally illustrate triangular shaped protrusions. FIGS. 9a-c illustrate triangular 14, inverse-triangular 15 and tear-drop-shaped 16 configurations respectively of the protrusion. FIGS. 10 a-c, illustrate square 77, hexagonal 18 and pentagonal 19 shaped protrusions respectively.

In some embodiments of the present invention, the scaffold is fabricated from material having sufficient rigidity to provide adequate radial strength to allow for use in arterial applications such as within the aorta. In one embodiment of the present invention, the scaffold is made from stainless steel. In other embodiments, the scaffold may be made from a material other than stainless steel, such as, for example, Nitinol. Typically, the longitudinal dimension of the stent is from about 30 mm to about 60 mm. Every vascular malformation requiring treatment with a stent is different, requiring the need for various different configurations of length of the stent. Hence, the dimension can also be less than 30 mm or greater than 60 mm. In one particular example the longitudinal length is about 45 mm.

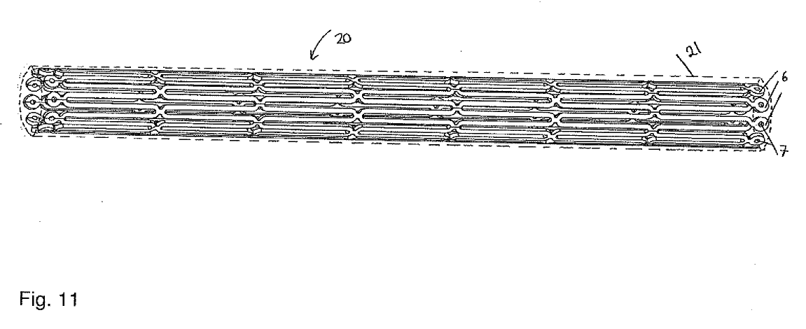

In an embodiment of the present invention, the stent is a covered stent 20 as illustrated in FIG. 11. A covered stent may also be referred to as a stent-graft or a conduit. In some embodiments, a cover 21 is attached to the scaffold at one or more portions of the outer surface of the scaffold. The cover 21 may also be attached to the inner surface of the scaffold. In some embodiments a cover may be attached to both the inner and the outer surface of the scaffold. Embodiments of the present invention, as described hereinabove, allow for the cover to be securely attached to the scaffold at one or more protrusions 6 and/or segments 10. The cover is secured to the scaffold by the application of an adhesive to the cavities 7, 17 in the scaffold. Typically, the adhesive is a biocompatible adhesive. In one embodiment of the present invention, the covered stent has a smooth outer surface. This provides the advantage of reducing the risk of restenosis after the stent has been implanted into the patient's body.

In one embodiment of the present invention, the cover is fabricated from, for example, expanded polytetrafluoroethylene (ePTFE), typically in conduit/tube form. In other embodiments, Polyurethane-based polymers which have a low durometer, having flexibility and the capability to stretch, are used to form the cover.

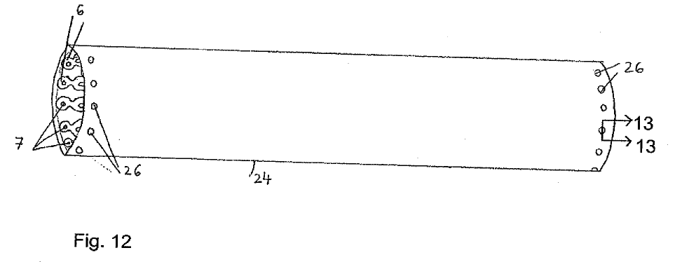

FIG. 12 is an illustration of an embodiment of a covered stent. In some embodiments, the ePTFE cover 24 is secured to the scaffold through the selective application of an adhesive through holes 26 created in the cover 24 using a laser. The laser may be a CO₂ laser. In alternate embodiments, other methods of perforation may be used to make the holes 26.

In one embodiment of the present invention, as shown for example in FIG. 12, the holes 26 are created such that they are substantially aligned with one or more of cavities 7, 17, defined by the protrusions 6 at the ends of the scaffold, and optionally segments 10 within the mesh structure of the scaffold. As illustrated in FIG. 12, the holes 26 are aligned with the cavities 7, at either end of the scaffold to ensure that, when attached to the scaffold, the ePTFE cover 24 extends across substantially the entire surface are of the scaffold.

In one embodiment of a method of manufacturing the covered stent, alcohol is applied on the periphery of the cover, after the cover has been applied overtop of the scaffold. The alcohol makes the cover substantially translucent, allowing the locations of the protrusions 6 and segments 10 to become visible through the cover 24, thereby allowing for the creation of one or more holes 26 in the cover aligned with cavities 7,17 using an appropriate laser or other perforation mechanism. These holes 26 can vary, for example, from about 0.05 mm in diameter to about 0.13 mm in diameter. In one embodiment, the diameter of the holes 26 is about 0.08 mm. Adhesive may be delivered to the site of the holes 26 in liquid form using amounts varying from about 0.5 ml to about 5 ml. The adhesive acts to secure the cover to the scaffold. The cavities 7, 17 defined by the protrusions 6 and/or segments 10 provide increased surface area for bonding the adhesive to the scaffold, thus increasing the strength of the bond between the scaffold and the cover.

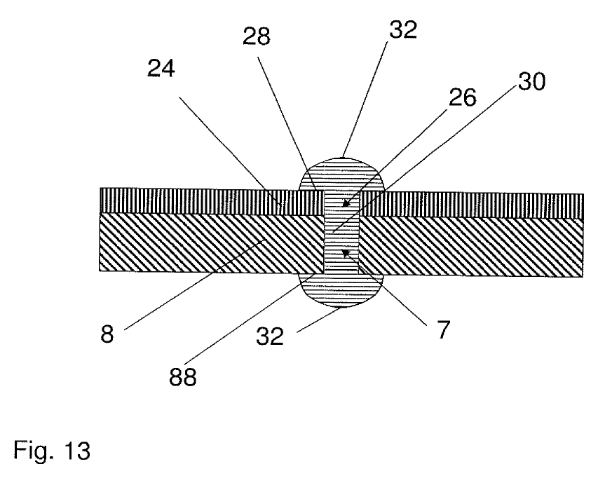

In some embodiments, as illustrated in FIG. 13, the adhesive upon drying forms a plug 30 that extends substantially from the exterior surface 28 of the cover 24 through the hole 26 and the cavity 7 to the inner surface 88 of the scaffold 8. In some such embodiments, the plug 30 forms a cap 32 at each of the surfaces 28, 88. In alternate embodiments, the plug 30 forms a cap 32 at one of the surfaces 28,88. The combination of the holes 26 and the cavities 7 provides for increased adhesion between the cover and the scaffold by allowing the adhesive to form a plug 30 which functions to clamp the cover and the scaffold to each other. In some embodiments an inner cover and an outer cover may be attached to the scaffold. In such embodiments, the adhesive forms a plug that functions to clamp both the inner and the outer covers to the scaffold. In some embodiments the cover may not comprise a hole and the adhesive forms a plug that extends for example from the inner surface of the scaffold to the outer surface of the scaffold. In some such embodiments, the plug forms a cap at one or more of the surfaces of the scaffold.

The amount of adhesive utilized is typically minimized to reduce complications that can arise when using a biocompatible adhesive. For example, if a large amount of adhesive is used, an oligomer can break off from the polymer chain inducing an inflammatory response within the patient. Another complication that can arise is the decrease in cell proliferation at locations where the adhesive is present. Hence, it may, in some embodiments, be beneficial to use small amounts of adhesive.

In one embodiment of the present invention, the adhesive is comprised of a solution containing a polymer and a solvent. Typically, the adhesive solution is substantially hygroscopic. Thus, in some embodiments, a process to facilitate drying is added after the adhesive has been applied to the covered stent. The solvent in the adhesive solution evaporates when heated or when placed in dry conditions for an extended period of time, allowing the polymer in the solution to act as a bonding agent. In one embodiment of the present invention, about 1 ml of adhesive is applied at the appropriate site(s) on the cover, for example using a dropper or a syringe. In one such embodiment, the adhesive solution comprises poly-l-lactic acid (PLLA), and the solvent utilized is chloroform. In an alternate embodiment, the polymer utilized is Polyurethane, and the solvent utilized is either 1,4-dioxane or Tetrahydrofurane or Dimethylacetamide. After the adhesive has been applied, the covered stent is heated at about 30 to about 60 degrees Celsius, more particularly at about 45 degrees Celsius, for a period of about 3-4 hours to dry the glue. In an alternative embodiment of the present invention, the covered stent can be placed in Nitrogen, Argon or Dry Air for a period of 3-4 hours to facilitate drying of the adhesive. In further embodiments, other bonding materials or means for attachment may be used.

In one particular example Poly-l-lactic acid (PLLA) with a molecular weight of 1,000,000 was used to glue the cover to the scaffold in an open air environment. The PLLA was dissolved in chloroform until the gel-like substance was liquid enough to be applied using a syringe, but still gel-like enough to avoid excessive slipping. A magnetic stirrer was used to dissolve the PLLA in chloroform, stirring at around 400 rpm for over 30 minutes. An ePTFE cover was placed over the stent, a low power CO₂ laser was used create the holes in the cover and glue was used on 5 protrusions to attach the cover to the stent from one side. After preparation, the covered stent was left to dry for several hours. It was then deployed, first using an 18 mm Balloon-In-Balloon (BIB) balloon and then using a 24 mm BIB balloon. The glue on four of the five protrusions remained intact and the protrusions remained well attached to the cover.

In an additional example, the adhesive used was polyurethane and a CO₂ laser was used to cut the cover. 5 protrusions were used to attach the cover to the scaffold. The adhesive in this sample remained intact on all five protrusions and the cover remained intact upon expansion using an 18 mm BIB balloon.

In one embodiment of the present invention, the cover is secured to a scaffold in a compressed state. The covered stent is operable to be inserted percutaneously while the covered stent is in the compressed state. The covered stent is then expanded once it has been inserted into a desired implantation site. To position the covered stent, standard X-ray fluoroscopy can be used to view the location of the stent as it is guided to the desired implantation site. In one embodiment of the present invention, radiopaque material may be incorporated with the stent to improve visibility of the stent under X-ray fluoroscopy. In another embodiment of the present invention, the radipaque material may be incorporated in the fluid solution that is used to expand the balloon.

In one embodiment of the present invention, the compressed stent is expanded using balloon angioplasty. In one such embodiment, the balloon is first folded and the stent is placed over it and crimped. The crimped balloon and stent are placed within a sheath and guided to the desired treatment site. The sheath is then removed and the balloon is deployed, expanding the stent in place. In one such embodiment the radial force utilized in expanding the stent is between about 101.325 kPa to about 202.65 kPa.

In one embodiment of the present invention the radius of the covered stent in the compressed configuration is between about 3 mm to about 4 mm. In one particular embodiment, the compressed radius of the stent and the cover is about 3.75 mm. The expanded radius of the stent can vary from about 6 mm to about 24 mm. The length of the cover can be from about 30 mm to about 60 mm, typically matching the length of the stent. In addition, the cover can have a wall thickness of between about 0.13 mm to about 0.25 mm. In one particular embodiment, the wall thickness of the cover is about 0.13 mm.

In one particular application, the implantation site is located within the aorta at a location of a coarctation. In some such applications, the covered stent is used to treat aortic coarctation within infants and adolescents, while minimizing or preventing bleeding from radial tears and minimizing risks of aneurysm formation or, later on, rupture. In further applications, the covered stent can be used in other areas where luminar support or vascular luminal support is required. In yet further applications, the covered stent may be used within any lumen within the body. Other specific vascular applications include use within carotid and cerebral vasculature. Other non-vascular applications of the covered stent include use within the pulmonary or the gastrointestinal regions.

Embodiments of the present invention thus provide a new structural design for a stent and a method for securely attaching a cover to the stent. The embodiments of the present invention allow for a cover to be secured to the stent while minimizing the shear forces acting on the cover and the likelihood of tears developing in the cover by minimizing the points of adhesion between the stent and the cover and maximizing the adhesion at these points.

The embodiments of the invention described above are intended to be exemplary only.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. All to publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A stent comprising:
a substantially elongated scaffold formed by a plurality of cells comprising a plurality of interlinked struts, said scaffold having two longitudinally opposed ends;
at least two protrusions, at least one of said at least two protrusions being structurally attached to at least one of said plurality of cells at one of said two longitudinally opposed ends, and at least another of said at least two protrusions being structurally attached to at least one of said plurality of cells at another of said two longitudinally opposed ends;
each of said at least two protrusions defining a cavity extending therethrough; and
a cover attached to said scaffold by an adhesive, said adhesive being located within the cavities to secure said cover to said scaffold at the protrusions.

2. The stent of claim 1 wherein said cover defines at least one hole, said at least one hole being aligned with at least one of said cavities, said adhesive extending through both said at least one of said cavities as well as said at least one hole.

3. The stent of claim 1, wherein said at least two protrusions extend integrally from said at least one of said plurality of cell to which they are structurally attached.

4. The stent of claim 1, wherein said at least two protrusions are attached to said at least one of said plurality of cells by an attachment means selected from the group consisting of welding and soldering.

5. The stent of claim 1, wherein the cavities extend substantially completely through the protrusions.

6. The stent of claim 1, wherein the cover is fabricated from expanded Polytetrafluoroethylene.

7. The stent of claim 1, wherein said adhesive is poly-l-lactic acid.

8. The stent of claim 13, wherein said adhesive is polyurethane.

9. The stent of claim 1, wherein at least one of said at least two protrusions is substantially annular.

10. The stent of claim 1, wherein one or more of said at least one protrusion structurally attached at one of said two longitudinally opposed ends comprises a plurality of protrusions and wherein said plurality of protrusions are circumferentially spaced apart from one another.

11. The stent of claim 11, wherein said plurality of protrusions are attached to every second cell from said plurality of cells at each of said two longitudinally opposed ends.

12. The stent of claim 1, wherein said plurality of cells comprises at least two adjacent cells having at least one segment disposed therebetween.

13. The stent of claim 10, wherein said segment has a substantially annular configuration.

14. The stent of claim 10, wherein said segment has substantially disk-shaped configuration.

15. The stent of claim 10, wherein said segment has a substantially ohm-shaped configuration.

16. The stent of claim 10, wherein said segment has a substantially sinusoidal configuration.

17. A stent comprising:
a substantially elongated scaffold formed by a plurality of cells comprising a plurality of interlinked struts, said scaffold having two longitudinally opposed ends;
a cover attached to said scaffold; and
at least two protrusions or segments each structurally attached to at least one of said plurality of cells, at least one of said protrusions or segments for securing one end of said cover, at least another of said protrusions or segments for securing another end of said cover, each of said at least two protrusions or segments defining a cavity extending therethrough;
wherein said cover is attached to said scaffold using an adhesive, said adhesive being located within the cavities to secure said cover to said scaffold at the protrusions or segments.
